(19) United States
(12) Patent Application Publication
(10) Pub. No.: US 2022/0049313 A1
(43) Pub. Date: Feb. 17, 2022

(54) PRIMER FOR DETECTING TUMOR MICROSATELLITE INSTABILITY, USE THEREOF AND KIT

(71) Applicant: ZHENGZHOU KODIA BIOTECHNOLOGY CO., LTD., Zhengzhou (CN)

(72) Inventors: Lulu ZHANG, Zhengzhou (CN); Xiankun LI, Zhengzhou (CN); Ge MENG, Zhengzhou (CN); Xiaoqiang TIAN, Zhengzhou (CN); Wenru XU, Zhengzhou (CN)

(21) Appl. No.: 17/178,813

(22) Filed: Feb. 18, 2021

(30) Foreign Application Priority Data
Aug. 11, 2020 (CN) 202010804220.5

(51) Int. Cl. C12Q 1/6886; C12Q 1/686

(52) U.S. Cl. CPC C12Q 1/6886; C12Q 2600/16; C12Q 1/686

(57) ABSTRACT

In the technical field of molecular diagnosis, a detection primer for detecting tumor microsatellite instability (MSI), use thereof, a kit and method for amplifying tumor microsatellite loci is disclosed. The detection primer includes one or more of microsatellite locus amplification primers selected from sequences shown as SEQ ID NOs: 1 to 26 and can simultaneously amplify a plurality of microsatellite loci in the same PCR system and perform multiplex fluorescent PCR to detect MSI, with no cross peak or overlapped peak among fluorescence peaks generated by electrophoresis of amplified products of microsatellite sites and with high detection efficiency and excellent sensitivity.

The Sequence Listing under document GAOWOBSEQUENCELISTING.txt, created Feb. 17, 2021 with 6,000 bytes is incorporated by reference.

### FIELD OF THE INVENTION

The present invention relates to the technical field of molecular diagnosis, and in particular to a primer for detecting tumor microsatellite instability (MSI), the use thereof and a kit.

### BACKGROUND OF THE INVENTION

A “Microsatellite” is a short tandem repeat (“STR”) DNA sequence present throughout the human genome in noncoding regions between genes or within genes, comprising single-nucleotide, dinucleotide, trinucleotide, or high nucleotide repeats, which have about 10-50 repeats. Compared with microsatellites in normal cells, the length of microsatellites in tumor cells change due to the insertion or deletion of repeat units, referred to as MSI. MSI is a condition of genetic hypermutability, or a predisposition to mutation.

A large number of studies have shown that MSI is caused by defects or deficiencies in mismatch repair (MMR) genes and is closely related to tumorigenesis. MSI has been clinically used as an important molecular marker for the prognosis of colorectal cancer and other solid tumors and the formulation of adjuvant therapeutic regimens. MSI has also been used to assist in the screening of Lynch syndrome.

According to literature reports, about 15% of patients with colorectal cancers have a microsatellite instability-high (MSI-H) phenomenon, the pathogenesis, prognosis and drug susceptibility of which are different from colorectal cancers characterized by microsatellite stability (MSS). In non-colorectal cancer solid tumors, there are also different proportions of MSI-H phenomenon. Solid tumors with different MSI status have significant differences in the response rate to pembrolizumab (Keytruda®).

Lynch syndrome, also known as hereditary non-polyposis colorectal cancer (HNPCC), is a dominant genetic disease caused by germline mutations in the MMR gene. More than 90% of patients with Lynch syndrome are characterized by MSI-H, and only about 15% of patients with sporadic colorectal cancer are characterized by MSI-H. Accordingly, MSI detection provides a clinical method to screen for Lynch syndrome.

In addition, colorectal cancer, endometrial cancer, gastric cancer, ovarian cancer and other Lynch syndrome-related tumors are common in patients with Lynch syndrome and family members thereof. Therefore, MSI detection is of great significance for screening not only the patients but also their family members.

On May 23, 2017, the FDA approved the PD-1 antibody drug pembrolizumab (Keytruda®) for the first time for the treatment of “dMMR/MSI-H” solid tumors. The approval marked a milestone in that this is the first anti-tumor therapy to differentiate based on molecular markers instead of tumor origin. In clinical trials, 86 patients with solid tumors characterized by MSI-H had an objective remission rate of 54% and a disease control rate of 72%.

When mis-match repair deficiency (dMMR) exists in tumor cells, this indicates that tumor cells have lost the ability to repair DNA replication errors, and a large number of mutations will accumulate in tumor cells, which will be accompanied by MSI features. Studies have shown that the more mutations carried by tumor cells the more neoantigens (truncated or flawed proteins or mutations) present (there is no neoantigen on the surface of a normal cell), which can be specifically recognized by the patient's own immune system with a higher probability that the immune system will specifically kill tumor cells. But often the reason that the immune system does not attack tumor cells is that tumor cells inhibit the killing effect of T cells through the PD-1/PD-L1 signaling pathway.

Keytruda® is a PD-1 monoclonal antibody that can block the PD-1/PD-L1 signaling pathway, thereby reducing the inhibitory effect of tumor cells on the killing effect of T cells. If there are a large number of neoantigens recognized by T cells on the surface of tumor cells at the same time, T cells can smoothly kill tumor cells and achieve better therapeutic effects. Therefore, in theory, those patients with dMMR/MSI-H solid tumors can further benefit after Keytruda® reduces the immunosuppressive effect. Thus, it is of great clinical significance to distinguish patients with MSI-H solid tumors through MSI detection.

Prior known MSI detection kits detect 5-7 loci but lacked specificity and sensitivity. There remains a need therefore for MSI detection methods with improved specificity and sensitivity and having a broader detection range (number of loci) compared to what was previously known in the art.

It is an object of the present invention to provide a primer for detecting tumor MSI having a broader range of detection and greater specificity and sensitivity.

Another object of the invention is to provide for use of the above-mentioned tumor MSI detection primer in related detection products and related kit products.

Still another object of the invention is to provide a method for amplifying tumor micro satellite loci using the detection primer to conduct PCR amplification on cancer and adjacent tissue, wherein mutations of microsatellite loci are detected by capillary electrophoresis.

Other objects of the invention will be evident to one skilled in the art.

### SUMMARY OF THE INVENTION

The present invention is directed to a primer for detecting tumor MSI, which can specifically detect up to 13 tumor microsatellite loci. The inventive primers can detect at the same time without affecting each other. An advantage of the present invention is that the detection range of the inventive primer has been greatly expanded compared with the prior known, common MSI kits for detecting 5-7 loci, thus improving the specificity and sensitivity of MSI detection. Moreover, the present inventive primer(s) covers all loci of common MSI detection products and can perform regular detection and high-sensitivity tests for double result evaluation. The present inventive primer eliminates ambiguous results in routine MSI detection.

To achieve the above objectives, the present invention provides the following technical solutions:

In one embodiment, the invention is a detection primer for detecting tumor MSI comprising one or more microsatellite locus amplification primers of:

upstream and downstream amplification primers at the NR27 locus shown as SEQ ID NOs: 1 to 2;

upstream and downstream amplification primers at the NR21 locus shown as SEQ ID NOs: 3 to 4;

upstream and downstream amplification primers at the NR22 locus shown as SEQ ID NOs: 5 to 6;

upstream and downstream amplification primers at the NR24 locus shown as SEQ ID NOs: 7 to 8;

upstream and downstream amplification primers at the NF26 locus shown as SEQ ID NOs: 9 to 10;

upstream and downstream amplification primers at the EWSR16 locus shown as SEQ ID NOs: 11 to 12;

upstream and downstream amplification primers at the Bat40 locus shown as SEQ ID NOs: 13 to 14;

upstream and downstream amplification primers at the BatR11 locus shown as SEQ ID NOs: 15 to 16;

upstream and downstream amplification primers at the Bat25 locus shown as SEQ ID NOs: 17 to 18;

upstream and downstream amplification primers at the Bat26 locus shown as SEQ ID NOs: 19 to 20;

upstream and downstream amplification primers at the NAV17 locus shown as SEQ ID NOs: 21 to 22;

upstream and downstream amplification primers at the Mono27 locus shown as SEQ ID NOs: 23 to 24; and

upstream and downstream amplification primers at the ELNF1 (17) locus shown as SEQ ID NOs: 25 to 26.

Preferably, a fluorescence marker may be ligated to the 5′-end of the upstream or downstream primers of the microsatellite locus amplification primers identified above; and/or the detection primer may include the sample ID locus amplification primers disclosed herein to avoid confusion of test samples.

In another embodiment, the invention is a kit comprising the detection primer of the invention and a fluorescent PCR reagent.

In still another embodiment, the invention is a method for amplifying tumor microsatellite loci using the detection primers of the invention to conduct PCR amplification on cancer and adjacent tissues, wherein mutations of microsatellite loci are detected by capillary electrophoresis.

Other embodiments of the invention will be evident to one skilled in the art based upon the disclosure set forth herein.

### BRIEF DESCRIPTION OF THE DRAWINGS

FIGS. 1 A and 1 B illustrate and compare the results of a sequencer analyzing sequence differences between a colorectal tumor tissue (tissue 1) verified as MSI-H by immunohistochemical ddMMR and a normal adjacent tissue thereof (tissue 2) obtained directly from the same patient (Patient sample 1), based on capillary electrophoresis, where FIG. 1 A shows the results obtained for tissue 1, and FIG. 1 B shows the results obtained for tissue 2;

FIGS. 2 A and 2 B illustrate and compare the results of a sequencer analyzing sequence differences between a colorectal tumor tissue verified as MSI-H by immunohistochemical ddMMR (tissue 1) and a normal adjacent tissue thereof (tissue 2), both obtained directly from the same patient (Patient sample 2), based on capillary electrophoresis, where FIG. 2 A shows the results obtained for tissue 1, and FIG. 2 B shows the results obtained for tissue 2;

FIGS. 3 A and 3 B illustrate and compare the results of a sequencer analyzing sequence differences between a colorectal tumor tissue verified as MSI-H by immunohistochemical ddMMR (tissue 1) and a normal adjacent tissue thereof (tissue 2) obtained directly from the same patient (Patient sample 3), based on capillary electrophoresis, where FIG. 3 A shows the results obtained for tissue 1, and FIG. 3 B shows the results obtained for tissue 2;

FIGS. 4 A and 4 B illustrate the results of a sequencer analyzing sequence differences between a colorectal tumor tissue verified as MSI-H by immunohistochemical ddMMR (tissue 1) and a normal adjacent tissue thereof (tissue 2) obtained from the same patient, based on capillary electrophoresis, using a commercially available foreign detection product (CAT# MD1641; Promega) product, where FIG. 4 A shows the results obtained for tissue 1, and FIG. 4 B shows the results obtained for tissue 2, when using the Promega product;

FIGS. 5 A and 5 B illustrate the results of a sequencer analyzing sequence differences between a colorectal tumor tissue verified as MSI-H by immunohistochemical ddMMR (tissue 1) and a normal adjacent tissue thereof (tissue 2) obtained from the same patient, based on capillary electrophoresis, using the detection primer of the present disclosure, where FIG. 5 A shows the results obtained for tissue 1, and FIG. 5 B shows the results obtained for tissue 2 when using the detection primer of the present disclosure.

### DETAILED DESCRIPTION OF THE INVENTION

The present invention is directed to a primer for detecting tumor MSI, the use thereof and a kit comprising the primer. The present invention is also directed to a method for amplifying tumor microsatellite loci, where the inventive detection primer is used to conduct PCR amplification on rectal cancer tissues and adjacent tissues thereof, mutations of microsatellite loci are detected by capillary electrophoresis, and the degree of MSI is analyzed instrumentally.

In one embodiment, a detection primer for detecting tumor MSI is provided, including one or more of the following 13 tumor microsatellite locus amplification primers:

upstream and downstream amplification primers at the NR27 locus shown as SEQ ID NOs: 1 to 2;

upstream and downstream amplification primers at the NR21 locus shown as SEQ ID NOs: 3 to 4;

upstream and downstream amplification primers at the NR22 locus shown as SEQ ID NOs: 5 to 6;

upstream and downstream amplification primers at the NR24 locus shown as SEQ ID NOs: 7 to 8;

upstream and downstream amplification primers at the NF26 locus shown as SEQ ID NOs: 9 to 10;

upstream and downstream amplification primers at the EWSR16 locus shown as SEQ ID NOs: 11 to 12;

upstream and downstream amplification primers at the Bat40 locus shown as SEQ ID NOs: 13 to 14;

upstream and downstream amplification primers at the BatR11 locus shown as SEQ ID NOs: 15 to 16;

upstream and downstream amplification primers at the Bat25 locus shown as SEQ ID NOs: 17 to 18;

upstream and downstream amplification primers at the Bat26 locus shown as SEQ ID NOs: 19 to 20;

upstream and downstream amplification primers at the NAV17 locus shown as SEQ ID NOs: 21 to 22;

upstream and downstream amplification primers at the Mono27 locus shown as SEQ ID NOs: 23 to 24; and

upstream and downstream amplification primers at the ELNF1 (17) locus shown as SEQ ID NOs: 25 to 26.

The present invention resolves the problems of few loci and poor detection sensitivity and specificity of existing detection kits and provides amplification primers for the above-described 13 tumor microsatellite loci, which may efficiently and specifically amplify sequences of target loci and improve the sensitivity of detection results.

Preferably, in another embodiment, a fluorescence marker may be ligated to the 5′-end of the upstream or downstream primers of the microsatellite locus amplification primers, and the fluorescence marker may be selected from fluorescent dyes, i.e., FAM™ (fluorescein), JOE™, or TAMRA™. Other fluorescent markers will be known to one skilled in the art.

Preferably, the detection primer may further include one or more of the following sample ID locus amplification primers to avoid confusion of test samples:

upstream and downstream amplification primers at the Penta E locus shown as SEQ ID NOs: 27 to 28;

upstream and downstream amplification primers at the Amel locus shown as SEQ ID NOs: 29 to 30; and

upstream and downstream amplification primers at the D165539 locus shown as SEQ ID NOs: 31 to 32.

In a specific embodiment of the present invention, sequences and fluorescence markers of detection primers are shown in Table 1:

TABLE 1

| | Micro- | | |
|---|---|---|---|
| Detection | satellite | Fluorescence | |
| primer | locus | marker | Sequence |
| MG3-6F-TMR | NR27 | 5′-TAMRA | taagatgcttgctaagact |
| MG3-6R | NR27 | | gtccaaattgcttgtctctgtgg |
| MG1-2F | NR21 | | ggtgtcgctggctctgtagtt |
| MG1-2R-Fam | NR21 | 5′-FAM | ctcgcgttaagtaagttgtaatgt |
| MG1-3F-Fam | NR22 | 5′-FAM | cccaaagctgtaattaagctcct |
| MG1-3R | NR22 | | gaaagtgccaaagctaagct |
| MG1-4F | NR24 | | gtctcgtcctgctagtaatgct |
| MG1-4R-Fam | NR24 | 5′-FAM | aagtgcccttgtataagaagct |
| MG1-5F-Fam | NF26 | 5′-FAM | tgtggggttgtgtaagaagttgt |
| MG1-5R | NF26 | | gaaatggcaagcaagttaagct |
| MG1-6F | EWSR16 | | pcaaaaagaaagggaagccctt |
| MG1-6R-Fam | EWSR16 | 5′-FAM | tgcttgctccctgagtact |
| MG1-7F | Bat40 | | gcaagctgtcctcaaggtaagt |
| MG1-7R-Fam | Bat40 | 5′-FAM | tggttgtgcttgtcctccttgt |
| MG2-1F | BatR11 | | gctgcaagtccaaagtgcaaatt |
| MG2-1R-Joe | BatR11 | 5′-Joe | tggaagtcftgttgtaagtctctcct |
| MG2-3F-Joe | Bat25 | 5′-Joe | tccaaagttggtccacttgct |
| MG2-3R | Bat25 | | ggttgtgt_gtagcgaagccgct |
| MG3-3F-TMR | Bat26 | 5′-TAMRA | ctccttgtaagaaagtgggtgtgt |
| MG3-3R | Bat26 | | gcaaattaagaaaggctcaaaaaagct |
| MG2-2F-Joe | NAV17 | | gttgtgtgccgtgtagtcgc |
| MG2-2R | NAV17 | 5′-Joe | cttggaaagttaagtcaaaaaagggt |
| MG3-2F | Mono27 | | tgtaatgtgtgttgtaagaaaaagcct |
| MG3-2R-TMR | Mono27 | 5′-TAMRA | gttggaagtgtaataagtggtcctct |
| MG3-4F-TMR | ELNF1 (17) | 5′-TAMRA | cctgcctgtgtgtctgtg_ctt |
| MG3-4R | ELNF1 (17) | | tgtagctggttggcaagtgtaaaaa |
| MG2-4F | Penta E | | tgtcaagaaatgtcaagtgcct |
| MG2-4R-Joe | Penta E | 5′-Joe | ttaagctaagaagaaaaaaaagcc |
| MG3-1F | Amel | | gcaaagaaagctgtaaaaagtgtt |
| MG3-1R-TMR | Amel | 5′-TAMRA | ctgtaagcctctgtaataagcct |
| MG3-5F | D16s539 | | gaagccagagagagaaagggtgctt |
| MG3-5R-TMR | D16s539 | 5′-TAMRA | aaagcgaaagtgtgtgcaagtgtt |

Also, an MSI classification standard is disclosed, as shown in Table 2:

TABLE 2

| | Proportion of | Number of |
|---|---|---|
| MSI | MSI markers | positive markers |
| MSI-H | >30% | ≥4/13 |
| MSI-L | <30% | <4/13 |
| MSS | 0% | 0/13 |

As used above, the detection result “MSI-H” means high MSI; the detection result “MSI-L” means low MSI; and the detection result “MSS” means microsatellite stability (no mutated loci).

In the present invention, tumor tissues verified as MSI-H by immunohistochemical ddMMR are used as detection objects, and normal adjacent tissues (MSI-Normal) of patients with MSI-H tumor tissue are used as controls. These tissues are subjected to multiplex fluorescent PCR-capillary electrophoresis using the detection primer of the present invention. Results show that the number of peaks of mutated loci in MSI-H tumor tissues increases, and the degree of instability is ≥4/13 (four mutated loci). The detection results are consistent with the immunohistochemical results.

Further, the present invention also uses a commercially available similar product as a control to detect the same tumor tissues diagnosed as MSI-H. As a result, the instability of the commercially available product is diagnosed as MSI-L, whereas the present invention can be correctly diagnosed as MSI-H, indicating that the detection primer of the present disclosure has higher specificity and makes the detection result more sensitive. Based on this, the present invention proposes use of the detection primer in the preparation of a kit for detecting tumor MSI or in PCR amplification of tumor MSI loci.

According to the above use, another embodiment of the present invention is directed to a kit for detecting tumor MSI, including the detection primer of the present invention and a fluorescent PCR reagent. Herein, the fluorescent PCR may preferably be multiplex fluorescent PCR.

Preferably, the PCR reagent may include nuclease-free deionized water and/or PCR Mix. In a specific embodiment, Multiplex PCR Mix may be used.

In yet another embodiment, the present invention further provides a method for amplifying tumor microsatellite loci, where the detection primer of the present disclosure is used to conduct PCR amplification on rectal cancer tissues and adjacent tissues thereof, mutations of microsatellite loci are detected by capillary electrophoresis, and the degree of MSI is analyzed instrumentally.

In a specific embodiment of the present disclosure, the PCR amplification may have the following program:

running denaturation at 95° C. for 10 min, 32 cycles of 94° C. for 20 s, 58° C. for 30 s, and 70° C. for 40 s, and finally 60° C. for 20 min.

The PCR system is shown in Table 3:

TABLE 3

| Component | Ratio | |
|---|---|---|
| 5× multiplex PCR Mix | 2 | μL |
| 5× Primer Mix | 2 | μL |
| Sample template | 1-5 | μL; total 1 ng |
| Nuclease-free deionized water | | Make up the volume to 10 μL |
| Total system | 10 | μL |

Herein, the Primer Mix may be prepared according to the following Table 4, and 6.4 μL of TE is added; the total volume is 20 μL;

TABLE 4

| Locus | NR-27 | | NR-21 | | NR-22 | | NR-24 | | NR-26 | | EWSR16 | | Bat-40 | | BatR11 | |
|---|---|---|---|---|---|---|---|---|---|---|---|---|---|---|---|---|
| Primer | 1 | μL | 1. | μL | 0.8 | μL | 1.2 | μL | 0.5 | μL | 1.2 | μL | 0.5 | μL | 0.6 | μL |
| volume | | | | | | | | | | | | | | | | |
| Final | 0.5 | μM | 0.5 | μM | 0.4 | μM | 0.6 | μM | 0.25 | μM | 0.6 | μM | 0.25 | μM | 0.3 | μM |
| concen- | | | | | | | | | | | | | | | | |
| tration | | | | | | | | | | | | | | | | |
| | | | | | | | | | ELNE1 | | | | | | | |
| Locus | Bat-25 | | Bat-26 | | NAV17 | | Mono-27 | | (17) | | Penta E | | Auld | | D16s539 | |
| Primer | 1 | μL | 1 | μL | 0.8 | μL | 1.2 | μL | 0.5 | μL | 1.2 | μL | 0.5 | μL | 0.6 | μL |
| volume | | | | | | | | | | | | | | | | |
| Final | 0.5 | μM | 0.5 | μM | 0.4 | μM | 0.6 | μM | 0.25 | μM | 0.6 | μM | 0.25 | μM | 0.3 | μM |
| concen- | | | | | | | | | | | | | | | | |
| tration | | | | | | | | | | | | | | | | |

It can be seen from the above technical solutions that: the detection primer of the present disclosure may simultaneously amplify a plurality of microsatellite loci in the same PCR system and perform multiplex fluorescent PCR to detect MSI with high detection efficiency and excellent sensitivity; there is no cross peak or overlapped peak among fluorescence peaks generated by electrophoresis of amplified products of microsatellite sites.

One skilled in the art will understand from the content of this application and appropriately improve the process parameters. Of particular note, all similar substitutions and alterations will be apparent to those skilled in the art, and they are all deemed to be included within the scope of the present disclosure. The detection primer, the use thereof and the kit, along with methods, provided by the present disclosure have been described in preferred embodiments. Obvious modifications or appropriate changes and combinations of the detection primer, the use, the kit and the methods described herein may be made without departing from the content, spirit and scope of the present invention in order to realize and apply the full potential of the technology set forth in the present disclosure.

The present invention adopts multiplex fluorescent PCR-capillary electrophoresis to detect MSI in human tumor cells. According to the conservation of sequences flanking the microsatellite DNA, 13 pairs of specific primers are designed and microsatellite DNA sequences at all loci are amplified by PCR. After the microsatellite loci are amplified by fluorescent PCR, a genome scan is conducted by capillary electrophoresis on a genetic analyzer, and the MSI is determined by detecting the fragment length. Images are collected and analyzed by using Genescan and Genetyper software, and the size of amplified fragments of microsatellite allelic variation is calculated.

The technical solutions of the present invention are applicable to sequencers and genetic analyzers such as ABI 310, 3100, ABI 3130 series, 3500 series, and 3730 series.

The primer for detecting tumor MSI, the use thereof, the kit, and methods set forth herein are further described in the non-limiting example embodiments below, although the invention is not restricted as such.

### Embodiment 1: Clinical Sample Detection

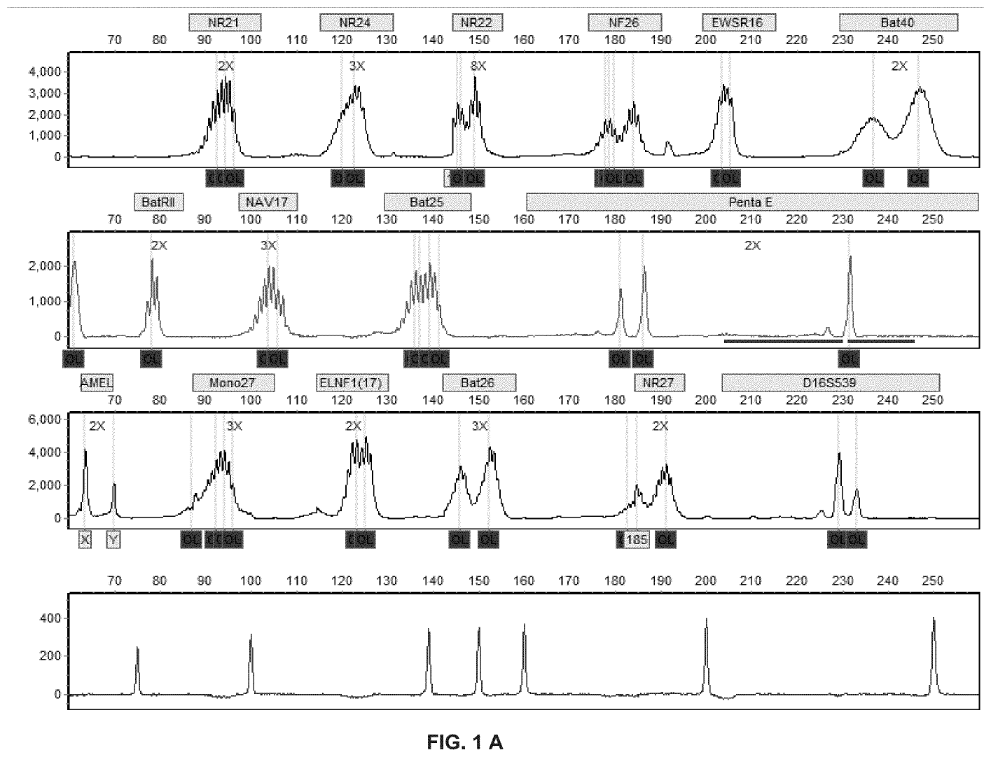

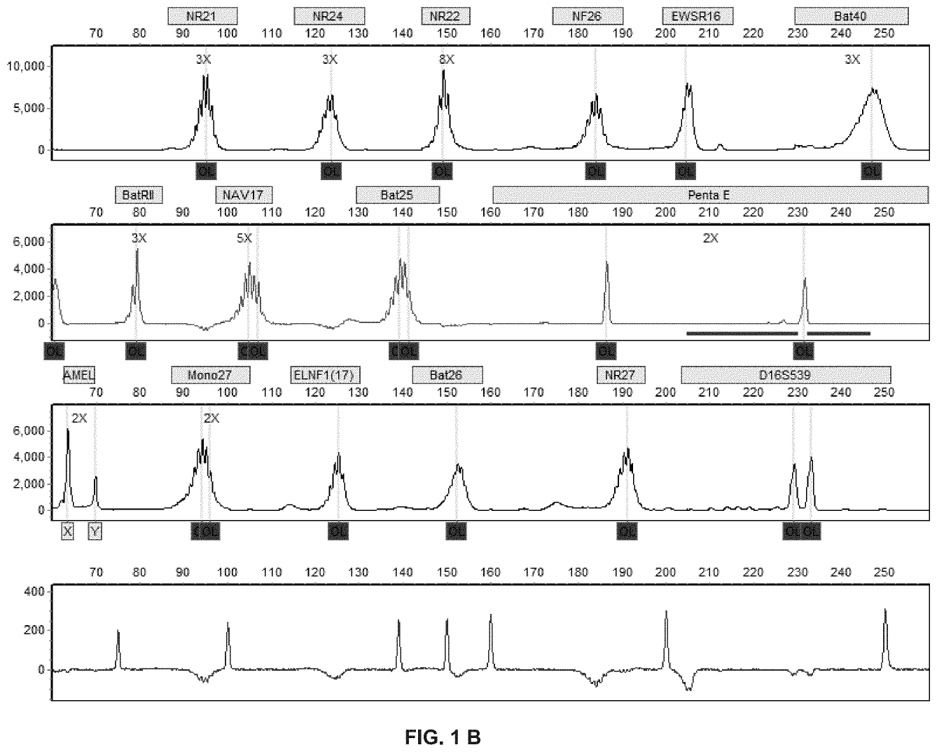

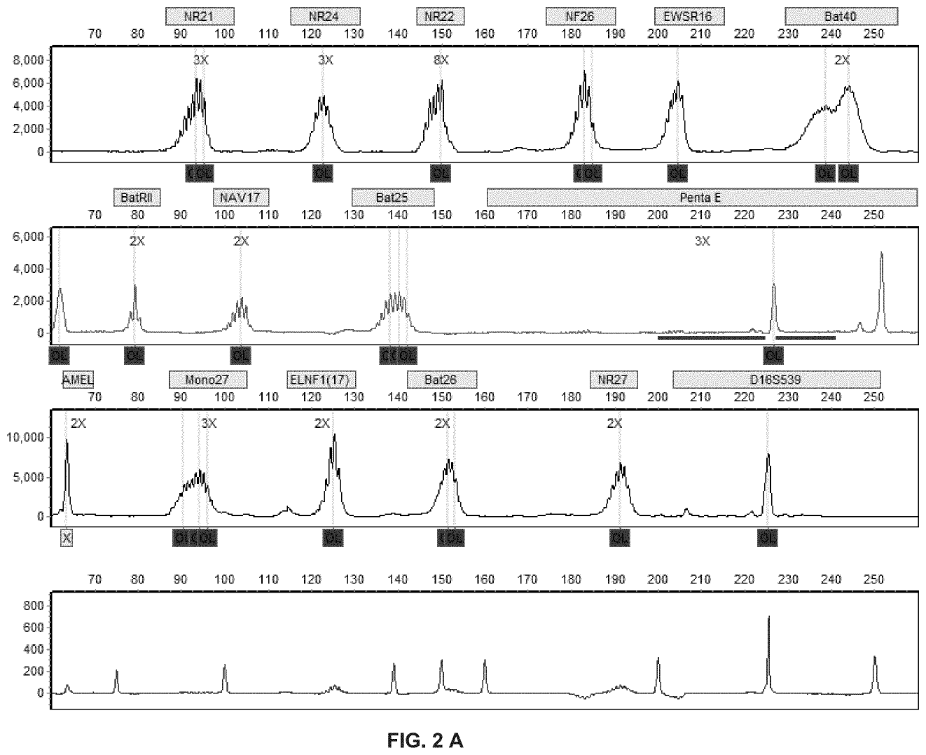

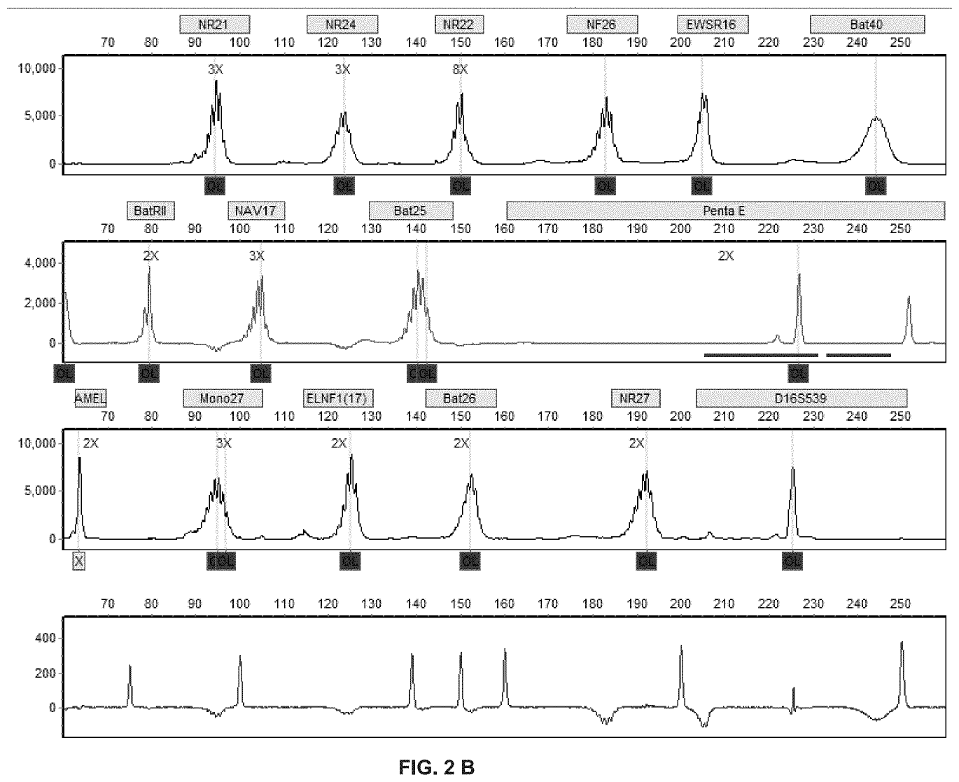

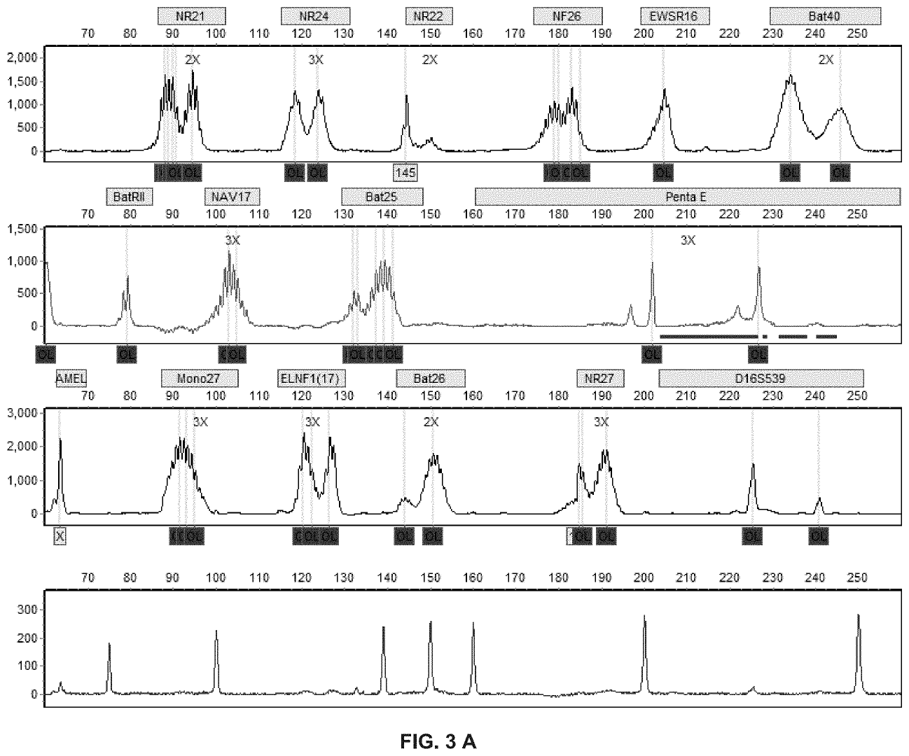

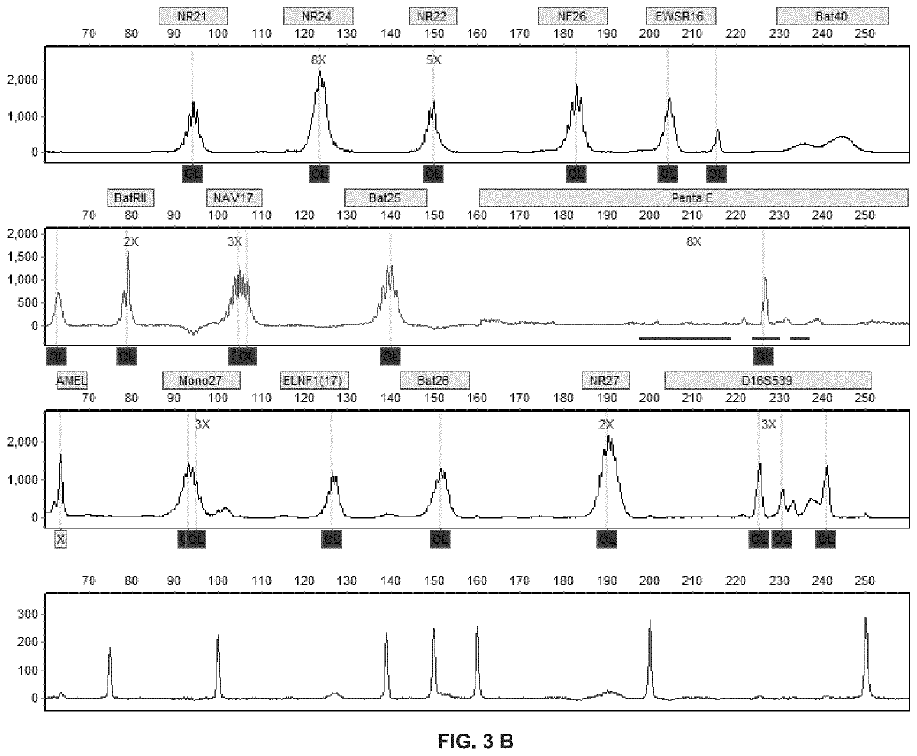

In this embodiment, samples were selected from three different patients, and each sample included the following two tissues:
tissue 1: a colorectal tissue verified as MSI-H by immunohistochemical ddMMR;
tissue 2: a normal adjacent tissue of a patient of tissue 1;
DNA templates were extracted from tissues 1 and 2, amplified by multiplex fluorescent PCR according to Tables 3 and 4, and detected by 3130 Genetic Analyzer (ABI's first generation of sequencer that analyzes sequence differences based on capillary electrophoresis). The results are shown in FIGS. 1 to 3.

From FIG. 1, there are mutations in more than 4 loci in tissue 1 of each of the three samples, and the number of peaks of the loci were different from that of the corresponding loci in the adjacent tissues. Thus, the degree of MSI is ≥4/13, which is diagnosed as MSI-H; the test results were consistent with the immunohistochemical results.

### Embodiment 2: Comparison with Similar Foreign Products in Detection

Commercially available similar product: Cat#MD1641, Promega; Sample:
tissue 1: a colorectal tissue verified as MSI-H by immunohistochemical ddMMR;
tissue 2: a normal adjacent tissue of a patient of tissue 1;

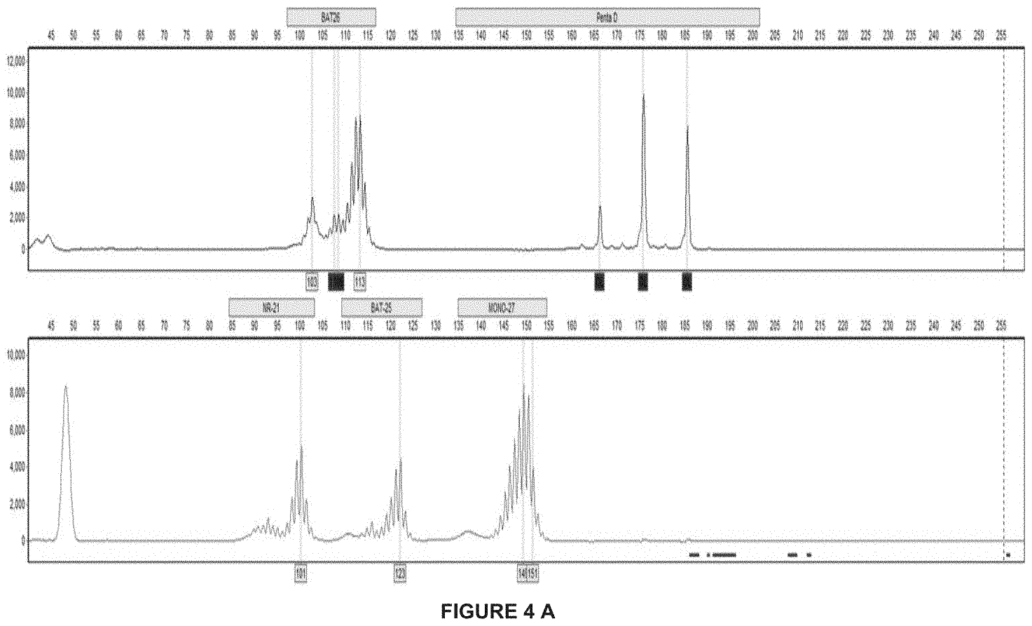

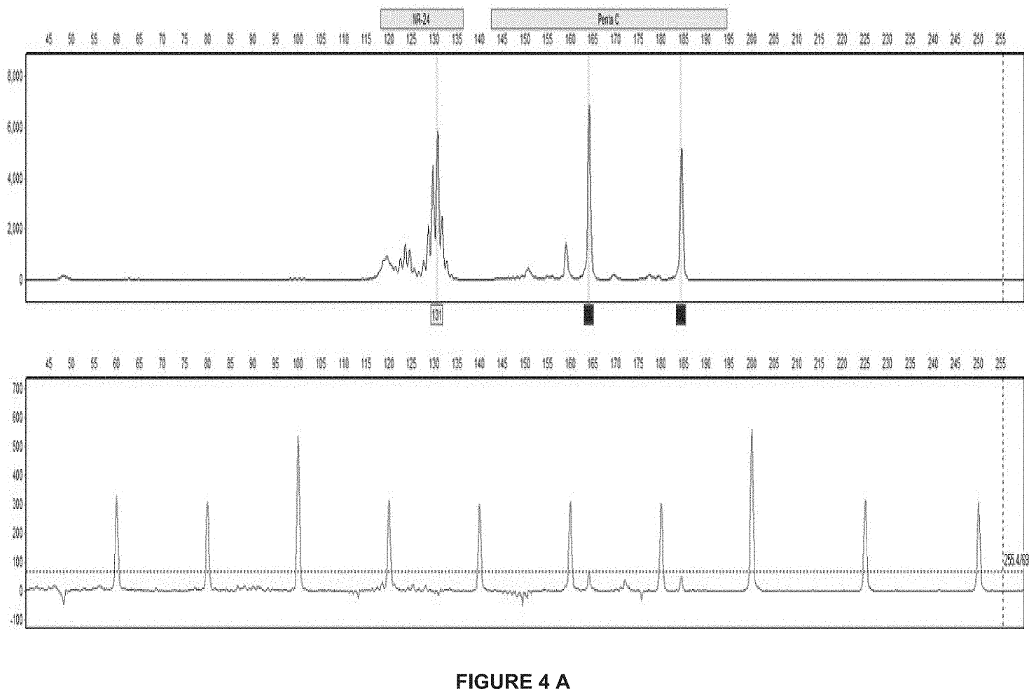

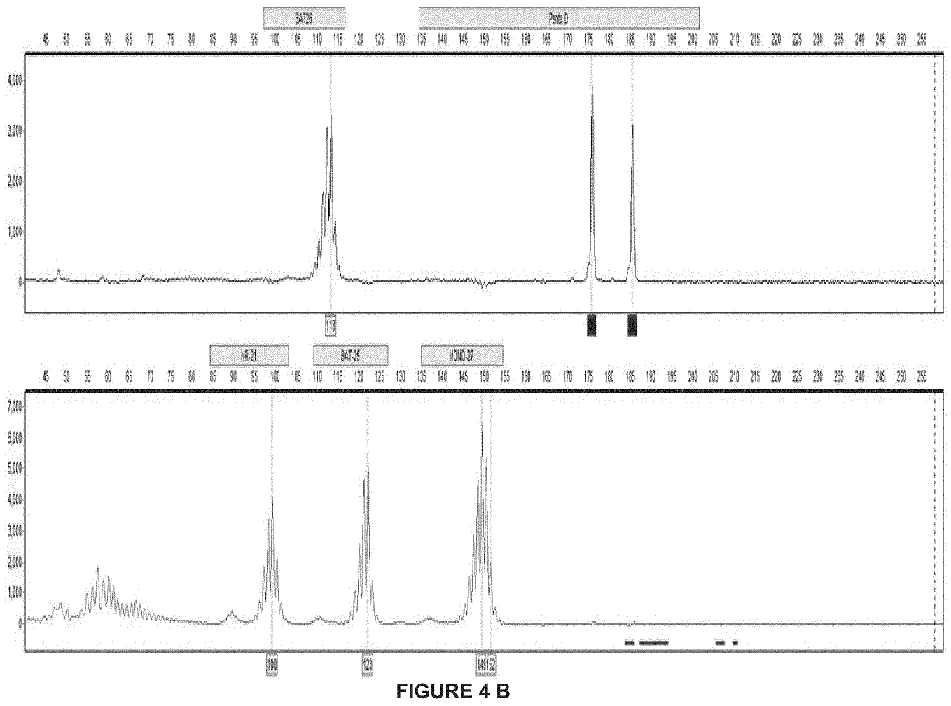

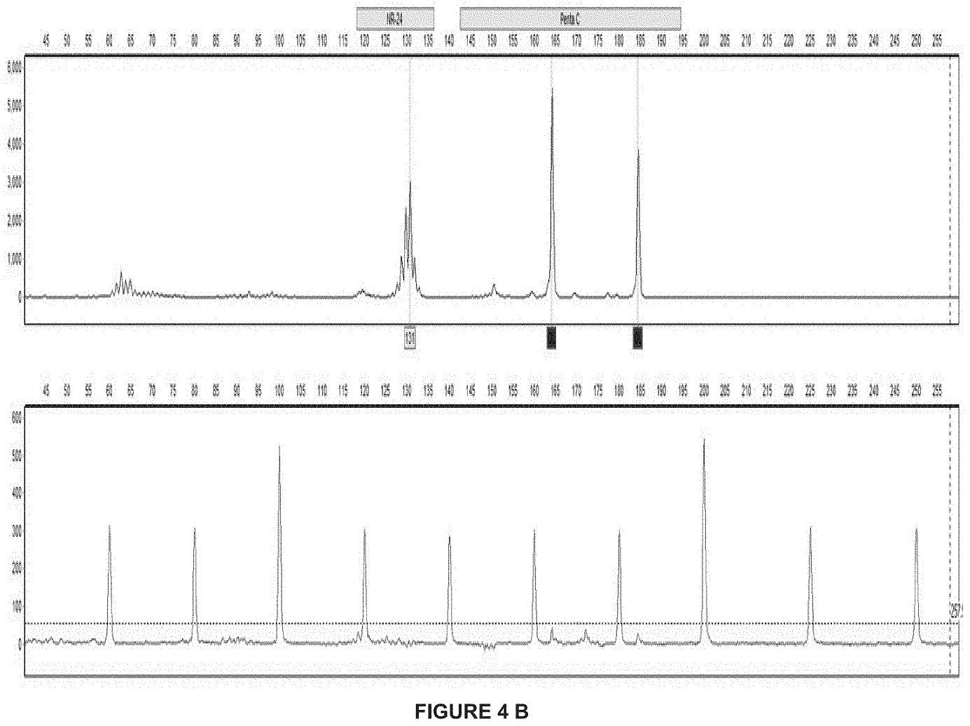

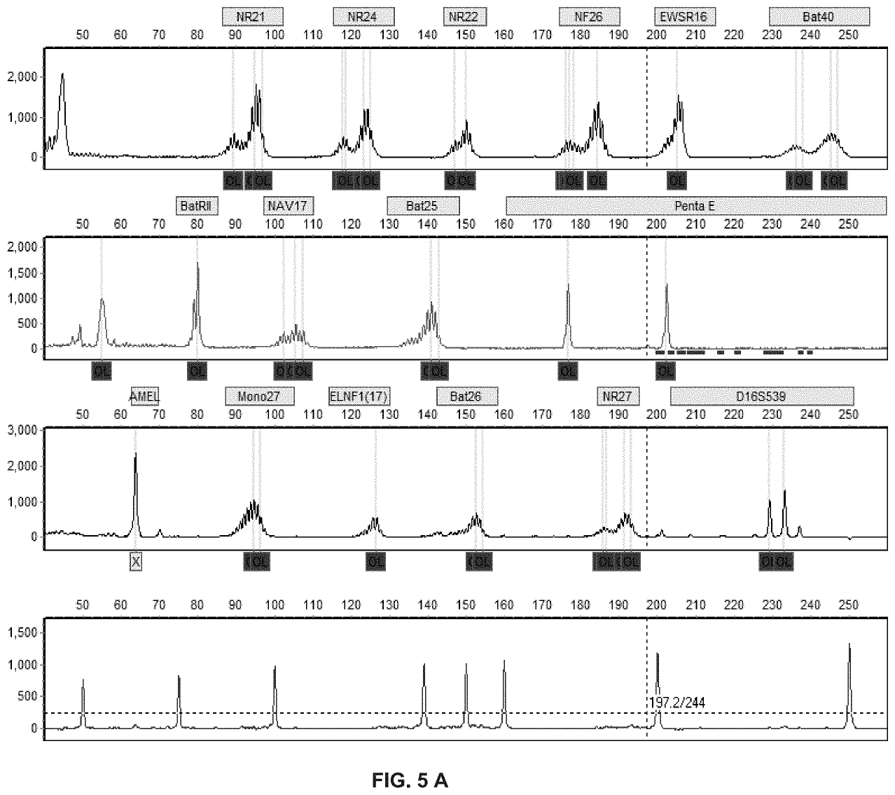

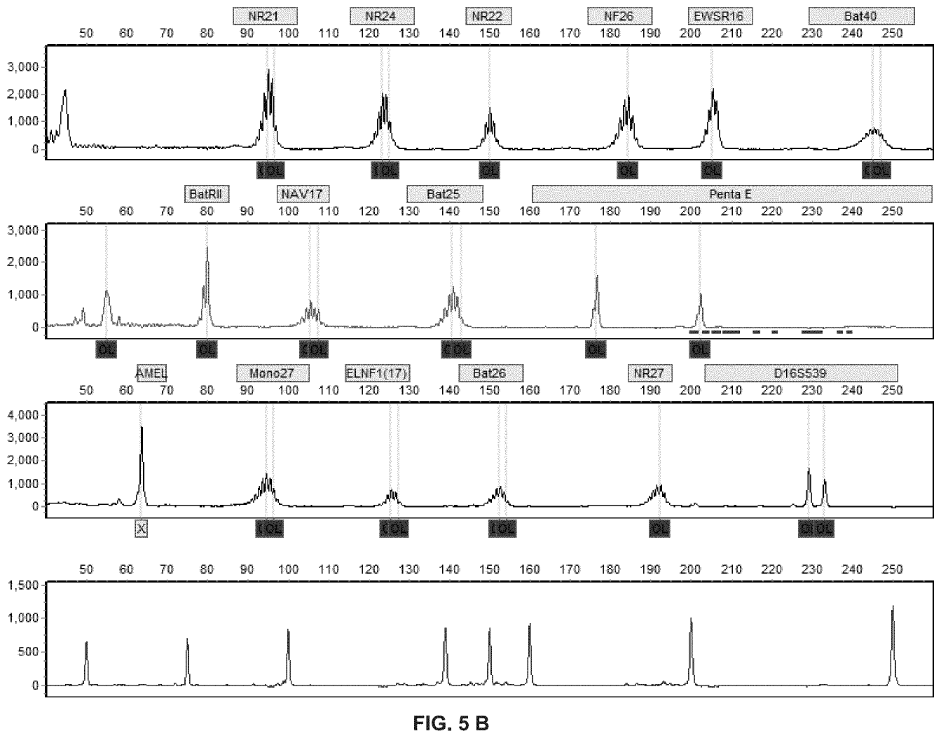

According to the detection method in Embodiment 1, detection primers of the present disclosure and the commercially available kit were used for detection; the results are shown in FIGS. 4 and 5.

From FIGS. 4 and 5, the present disclosure uses a commercially available similar foreign product and the detection primer of the present disclosure to detect the same sample tissue. The results show that the number of positive markers of Promega is 5/5, and the diagnosis result is MSI-H; the number of positive markers of the detection primer of the present disclosure is 11/13, and the diagnosis result is also MSI-H, indicating that the detection primer of the present disclosure has reached the detection level of foreign kits.

The foregoing examples set forth preferred embodiments of the present invention. It should be noted, however, that several changes, improvements and/or modifications are within the expertise of those of ordinary skill in the art without departing from the principles of the present invention. Such changes, improvements and/or modifications area contemplated by the present invention and are regarded as within the protection scope of the present disclosure.

What is claimed is:

1. A primer for detecting tumor microsatellite instability (MSI), comprising one or more of the following microsatellite locus amplification primers:
upstream and downstream amplification primers at the NR27 locus shown as SEQ ID NOs: 1 to 2;
upstream and downstream amplification primers at the NR21 locus shown as SEQ ID NOs: 3 to 4;
upstream and downstream amplification primers at the NR22 locus shown as SEQ ID NOs: 5 to 6;
upstream and downstream amplification primers at the NR24 locus shown as SEQ ID NOs: 7 to 8;
upstream and downstream amplification primers at the NF26 locus shown as SEQ ID NOs: 9 to 10;
upstream and downstream amplification primers at the EWSR16 locus shown as SEQ ID NOs: 11 to 12;
upstream and downstream amplification primers at the Bat40 locus shown as SEQ ID NOs: 13 to 14;
upstream and downstream amplification primers at the BatR11 locus shown as SEQ ID NOs: 15 to 16;
upstream and downstream amplification primers at the Bat25 locus shown as SEQ ID NOs: 17 to 18;
upstream and downstream amplification primers at the Bat26 locus shown as SEQ ID NOs: 19 to 20;
upstream and downstream amplification primers at the NAV17 locus shown as SEQ ID NOs: 21 to 22;
upstream and downstream amplification primers at the Mono27 locus shown as SEQ ID NOs: 23 to 24; and
upstream and downstream amplification primers at the ELNF1 (17) locus shown as SEQ ID NOs: 25 to 26.

2. The detection primer according to claim 1, wherein a fluorescence marker is ligated to the 5′-end of the upstream or downstream primers of the microsatellite locus amplification primers.

3. The detection primer according to claim 2, wherein the fluorescence marker is FAM, Joe, or TAMRA.

4. The detection primer according to claim 1, further comprising one or more of the following sample ID locus amplification primers:
upstream and downstream amplification primers at the Penta E locus shown as SEQ ID NOs: 27 to 28;
upstream and downstream amplification primers at the Amel locus shown as SEQ ID NOs: 29 to 30; and
upstream and downstream amplification primers at the D16S539 locus shown SEQ ID NOs: 31 to 32.

5. The detection primer according to claim 2, further comprising one or more of the following sample ID locus amplification primers:
upstream and downstream amplification primers at the Penta E locus shown as SEQ ID NOs: 27 to 28;
upstream and downstream amplification primers at the Amel locus shown as SEQ ID NOs: 29 to 30; and
upstream and downstream amplification primers at the D16S539 locus shown as SEQ ID NOs: 31 to 32,

6. The detection primer according to claim 3, further comprising one or more of the following sample ID locus amplification primers:
upstream and downstream amplification primers at the Penta E locus shown as SEQ ID NOs: 27 to 28;
upstream and downstream amplification primers at the Amel locus shown as SEQ ID NOs: 29 to 30; and
upstream and downstream amplification primers at the D16S539 locus shown as SEQ ID NOs: 31 to 32.

7. A kit for detecting tumor MSI, comprising the detection primer according to claim 1 and a fluorescent PCR reagent.

8. A kit for detecting tumor MSI, comprising the detection primer according to claim 2 and a fluorescent PCR reagent.

9. A kit for detecting tumor MSI, comprising the detection primer according to claim 3 and a fluorescent PCR reagent.

10. A kit for detecting tumor MSI, comprising the detection primer according to claim 4 and a fluorescent PCR reagent.

11. The kit according to claim 7, wherein the PCR reagent comprises nuclease-free deionized water and/or PCR Mix.

12. The kit according to claim 8, wherein the PCR reagent comprises nuclease-free deionized water and/or PCR Mix.

13. The kit according to claim 9, wherein the PCR reagent comprises nuclease-free deionized water and/or PCR Mix.

14. The kit according to claim 10, wherein the PCR reagent comprises nuclease-free deionized water and/or PCR Mix.

15. A method for amplifying tumor microsatellite loci, wherein the detection primer according to claim 1 is used to conduct PCR amplification on rectal cancer tissues and adjacent tissues thereof, and mutations of microsatellite loci are detected by capillary electrophoresis.

16. A method for amplifying tumor microsatellite loci, wherein the detection primer according to claim 2 is used to conduct PCR amplification on rectal cancer tissues and adjacent tissues thereof, and mutations of microsatellite loci are detected by capillary electrophoresis.

17. A method for amplifying tumor microsatellite loci, wherein the detection primer according to claim 3 is used to conduct PCR amplification on rectal cancer tissues and adjacent tissues thereof, and mutations of microsatellite loci are detected by capillary electrophoresis.

18. A method for amplifying tumor microsatellite loci, wherein the detection primer according to claim 4 is used to conduct PCR amplification on rectal cancer tissues and adjacent tissues thereof, and mutations of microsatellite loci are detected by capillary electrophoresis.

19. The method according to claim 15, wherein the PCR amplification is conducted under the following program:
running denaturation at 95° C. for 10 min, 32 cycles of 94° C. for 20 s, 58° C. for 30 s, and 70° C. for 40 s, and finally 60° C. for 20 min.

20. The method according to claim 15, wherein a PCR system for use in conducting the PCR amplification comprises: 5×Multiplex PCR mix in an amount of 2 μL, 5×Primer Mix in an amount of 2 μL, Sample template 1-5 μL up to a total 1 ng, nuclease-free deionized water in an amount sufficient to achieve a volume for the PCR system of 10 μL.


SEQUENCE LISTING (32 sequences)

SEQ ID NO: 1 - DNA, 21 bp, Artificial Sequence
  MG3-6F-TMR
taagcttgcttgctaagctct

SEQ ID NO: 2 - DNA, 23 bp, Artificial Sequence
  MG3-6R
gtccaaattgcttgtctctgtgg

SEQ ID NO: 3 - DNA, 21 bp, Artificial Sequence
  MG1-2F
ggtgtcgctggctctgtagtt

SEQ ID NO: 4 - DNA, 24 bp, Artificial Sequence
  MG1-2R-Fam
ctcgcgttaagtaagttgtaatgt

SEQ ID NO: 5 - DNA, 23 bp, Artificial Sequence
  MG1-3F-Fam
cccaaagctgtaattaagctcct

SEQ ID NO: 6 - DNA, 20 bp, Artificial Sequence
  MG1-3R
gaaagtgccaaagctaagct

SEQ ID NO: 7 - DNA, 23 bp, Artificial Sequence
  MG1-4F
gtctcgtcctgctctgtaatgct

SEQ ID NO: 8 - DNA, 23 bp, Artificial Sequence
  MG1-4R-Fam
aagtgcccttgtcttaagaagct

SEQ ID NO: 9 - DNA, 23 bp, Artificial Sequence
  MG1-5F-Fam
tgtggggttgtgtaagaagttgt

SEQ ID NO: 10 - DNA, 22 bp, Artificial Sequence
  MG1-5R
gaaatggcaagcaagttaagct

SEQ ID NO: 11 - DNA, 23 bp, Artificial Sequence
  MG1-6F
gtcaaaaagaaagggaagccctt

SEQ ID NO: 12 - DNA, 21 bp, Artificial Sequence
  MG1-6R-Fam
tgcttgctccctgctgtctct

SEQ ID NO: 13 - DNA, 22 bp, Artificial Sequence
  MG1-7F
gcaagctgtcctcaaggtaagt

SEQ ID NO: 14 - DNA, 22 bp, Artificial Sequence
  MG1-7R-Fam
tggttgtgcttgtcctccttgt

SEQ ID NO: 15 - DNA, 23 bp, Artificial Sequence
  MG2-1F
gctgcaagtccaaagtgcaaatt

SEQ ID NO: 16 - DNA, 26 bp, Artificial Sequence
  MG2-1R-Joe
tggaagtcttgttgtaagtctctcct

SEQ ID NO: 17 - DNA, 22 bp, Artificial Sequence
  MG2-3F-Joe
tccaaagttggtccctcttgct

SEQ ID NO: 18 - DNA, 22 bp, Artificial Sequence
  MG2-3R
ggttgtgtgtagcgaagccgct

SEQ ID NO: 19 - DNA, 24 bp, Artificial Sequence
  MG3-3F-TMR
ctccttgtaagaaagtgggtgtgt

SEQ ID NO: 20 - DNA, 27 bp, Artificial Sequence
  MG3-3R
gcaaattaagaaaggctcaaaaaagct

SEQ ID NO: 21 - DNA, 20 bp, Artificial Sequence
  MG2-2F-Joe
gttgtgtgccgtgtagtcgc

SEQ ID NO: 22 - DNA, 26 bp, Artificial Sequence
  MG2-2R
cttggaaagttaagtcaaaaaagggt

SEQ ID NO: 23 - DNA, 27 bp, Artificial Sequence
  MG3-2F
tgtaatgtgtgttgtaagaaaaagcct

SEQ ID NO: 24 - DNA, 26 bp, Artificial Sequence
  MG3-2R-TMR
gttggaagtgtaataagtggtcctct

SEQ ID NO: 25 - DNA, 21 bp, Artificial Sequence
  MG3-4F-TMR
cctgcctgtgtgtctgtgctt

SEQ ID NO: 26 - DNA, 25 bp, Artificial Sequence
  MG3-4R
tgtagctggttggcaagtgtaaaaa

SEQ ID NO: 27 - DNA, 22 bp, Artificial Sequence
  MG2-4F
tgtcaagaaatgtcaagtgcct

SEQ ID NO: 28 - DNA, 24 bp, Artificial Sequence
  MG2-4R-Joe
ttaagctaagaagaaaaaaaagcc

SEQ ID NO: 29 - DNA, 24 bp, Artificial Sequence
  MG3-1F
gcaaagaaagctgtaaaaagtgtt

SEQ ID NO: 30 - DNA, 23 bp, Artificial Sequence
  MG3-1R-TMR
ctgtaagcctctgtaataagcct

SEQ ID NO: 31 - DNA, 25 bp, Artificial Sequence
  MG3-5F
gaagccagagagagaaagggtgctt

SEQ ID NO: 32 - DNA, 24 bp, Artificial Sequence
  MG3-5R-TMR
aaagcgaaagtgtgtgcaagtgtt